(19) United States
(12) Patent Application Publication
(10) Pub. No.: US 2026/0212987 A1
(43) Pub. Date: Jul. 23, 2026

(54) Prehabilitation Tool

(71) Applicant: C Digital Healthcare Ltd., London (GB)

(72) Inventor: Tarannum Rampal, London (GB)

(21) Appl. No.: 19/034,689

(22) Filed: Jan. 23, 2025

(51) Int. Cl. G16H 20/30; G16H 10/60; G16H 20/60

(52) U.S. Cl. CPC G16H 20/30; G16H 10/60; G16H 20/60

(57) ABSTRACT

A computer implemented method and/or system for providing a prehabilitation program for a patient prior to undergoing a medical treatment or procedure is provided. Health information of the patient and information relating to the medical treatment or procedure are received at a processor. This may be a user-based processor or a server-based processor. The health information is indicative of a current state of health for the patient. At the processor or another processor, the prehabilitation program for the patient is determined based on the received health information and the information relating to the medical treatment or procedure.

### FIELD OF THE DISCLOSURE

The disclosure concerns a computer implemented prehabilitation program.

### BACKGROUND TO THE DISCLOSURE

Prehabilitation is a multi-modal concept in modern medicine. The aim of prehabilitation is to optimize a patient prior to major medical intervention, for instance cancer treatment or a surgical procedure. This may assist in making the patient ready for the treatment or procedure. Scientific study of prehabilitation has shown it to have overall benefit in patient outcomes. However, these benefits appear to depend on personalization of the program and/or dynamically updating of the program as the patient progresses.

In view of these issues, expanding the provision of prehabilitation has been challenging.

### SUMMARY OF THE DISCLOSURE

Against this background, there is provided a computer implemented method and a computer system in accordance with the disclosed embodiments.

In embodiments, there is provided a computer implemented method for providing a prehabilitation program for a patient prior to undergoing a medical treatment or procedure. The method comprises: receiving at a processor, health information of the patient and information relating to the medical treatment or procedure, the health information being indicative of a current state of health for the patient; and determining, at the processor, the prehabilitation program for the patient based on the received health information and the information relating to the medical treatment or procedure.

In embodiments, the health information comprises information indicative of a current state of physical and mental health for the patient and wherein the prehabilitation program comprises a level of support to be provided to the patient.

In embodiments, the prehabilitation program comprises an exercise program and/or a nutrition program.

In embodiments, the step of determining comprises: determining upper and lower limits of daily protein intake.

In embodiments, the received health information comprises an indication of whether the patient can perform one or more specific activities independently and without pain and/or discomfort, and wherein the step of determining comprises selecting one or more exercises from a set of exercises for the exercise program, the one or more exercises being selected based on the indication of whether the patient can perform the one or more specific activities.

In embodiments, the specific activities comprise one or more of: sitting and standing from a chair, extending arms over head, laying down flat on the floor and standing up and/or wherein the set of exercises comprise one or more of: shoulder press; bridge; arm lift; and elbow push.

In embodiments, the exercise program comprises a circuit training prescription, at least one parameter of the circuit training prescription being set based on the received health information.

In embodiments, the received health information comprises a number of sit to stand movements that the patient can perform independently and without pain and/or discomfort within a 30 second period, and wherein the at least one parameter of the circuit training prescription is set based on the received number of sit to stand movements.

In embodiments, the at least one parameter of the circuit training prescription comprises a number of sit to stand exercises and wherein the number of sit to stand exercises is set based on whether the received number of sit to stand movements exceeds one or more thresholds.

In embodiments, the exercise program comprises a circuit training prescription irrespective of the medical treatment or procedure.

In embodiments, the circuit training prescription comprises one or more of: a hip knees up exercise; a shoulder press exercise; a lateral raise exercise; a knee extension exercise; a knee flexion exercise; a rowing exercise; a hip extension exercise; a hip abduction exercise; and a wall press exercise.

In embodiments, the exercise program comprises one of more condition-specific exercises, the one of more condition-specific exercises comprising one or more of: a pelvic floor exercise; a bracing exercise; a side plank exercise; a plank exercise; a bridge exercise; a shoulder shrug exercise; a shoulder circling exercise; a bent arm forward exercise; a bent arm sideways exercise; a back scratching exercise; a winging it exercise; a wall climbing exercise; an arm lift exercise; and an elbow push exercise, the one of more condition-specific exercises being selected based on the received information relating to the medical treatment or procedure.

In embodiments, the received health information comprises data from a multi-minute walking test undertaken by the patient, and wherein the step of determining comprises setting one or more cardiovascular exercises for the exercise program based on the data from the multi-minute walking test.

In embodiments, the exercise program comprises one or more of: physical exercises; mobility exercises; and breathing exercises.

In embodiments, the health information comprises an indication of a height and/or weight for the patient, and wherein the nutrition program comprises a recommended protein intake range that is set based on the received indication of a height and/or weight for the patient.

In embodiments, the method further comprises: presenting the prehabilitation program to the patient by the processor.

In embodiments, the method further comprises: monitoring performance of the prehabilitation program by the patient at the processor.

In embodiments, the step of monitoring is based on user-input information and/or sensor-based information and/or wherein the method further comprises adjusting the prehabilitation program by the processor based on the monitoring.

In embodiments, there is provided a computer system for providing a prehabilitation program for a patient prior to undergoing a medical treatment or procedure, the computer system comprising at least one processor, configured to: receive health information of the patient and information relating to the medical treatment or procedure, the health information being indicative of a current state of health for the patient; and determine the prehabilitation program for the patient based on the received health information and the information relating to the medical treatment or procedure.

In embodiments, there is provided a computer system for providing a prehabilitation program for a patient prior to undergoing a medical treatment or procedure, the computer system comprising: a user-based processor, configured to receive health information of the patient, the health information being indicative of a current state of health for the patient; and a server-based processor, configured to receive the health information from the user-based processor, to receive information relating to the medical treatment or procedure and to determine the prehabilitation program for the patient based on the received health information and the information relating to the medical treatment or procedure.

### BRIEF DESCRIPTION OF THE DRAWINGS

The invention may be put into practice in a number of ways, and preferred embodiments will now be described by way of example only and with reference to the accompanying drawings, in which:

FIG. 1 shows a schematic flowchart of a computer implemented method according to an embodiment;

FIG. 2 shows a schematic flowchart of a computer implemented method according to an embodiment; and

FIG. 3 shows a computer system according to an embodiment.

### DETAILED DESCRIPTION OF EMBODIMENTS

The disclosure provides a computer-based approach for providing a prehabilitation program for a patient prior to undergoing a medical treatment or procedure. Health information of the patient (indicative of a current state of health for the patient) and information relating to the medical treatment or procedure are obtained at a processor (or processor system). Then, the processor (or processor system) determines a prehabilitation program for the patient based on the received health information and the information relating to the medical treatment or procedure.

This approach provides a technology platform that may address challenges that have so far restricted wide scale adoption of prehabilitation. The received health information allows an assessment of a baseline level of the patient, such that a prehabilitation program that is personalized for that individual patient may be determined, particularly taking into consideration clinical and/or environmental circumstances that may impact the patient.

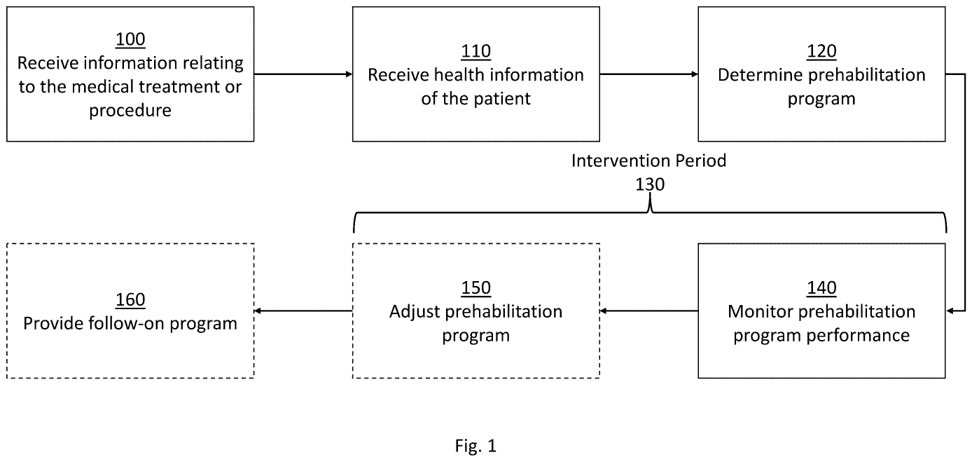

Referring first to FIG. 1, there is shown a schematic flowchart of a computer implemented method for providing prehabilitation according to an embodiment. The method may be implemented at a processor or processor system, for example in a computer server, which will generically be termed a system herein.

In a first step 100, the system (or specifically, a processor of the system) receives information relating to the medical treatment or procedure. For example, this may include one or more of: information about the illness or condition for which the patient is receiving treatment; information about the type of treatment or procedure (in this context, the term “procedure” may include a surgical operation) which the patient will be receiving; information about the criteria for receiving a treatment or procedure; and patient desired outcomes from the treatment or procedure. This may define the type of prehabilitation which the patient should receive.

In a second step 110, the system (or specifically, a processor of the system) receives health information of the patient. The health information is indicative of a current state of health for the patient. Such health information may comprise information indicative of a current state of physical and/or mental health for the patient, for instance in terms of one or more of: physical fitness; wellbeing and mental preparedness; and nutritional environment. The health information may encompass current state of the patient and/or personal circumstances of the patient.

One example of such information may include physical (and/or clinical) characteristics of the patient, for instance one or more of: height; weight; blood pressure; and a result of a medical test on the patient (for example, a blood test). Another example of such information may be an indication of whether the patient can perform one or more specific activities independently and without pain and/or discomfort. For instance, the information may comprise a number of sit to stand movements that the patient can perform independently and without pain and/or discomfort within a 30 second period and/or data from a multi-minute (for instance, 6 minute) walking test undertaken by the patient. Additional examples of specific activities may include one or more of: sitting and standing from a chair; extending arms over head; laying down flat on the floor and standing up. Further examples of health information may include one or more of: mental condition; nutritional intake and/or routine. Any of these examples may be used individually or in combination.

A key purpose of collecting the health information is for patient baseline assessment. Advantageously, the health information is provided to the system via an application used by the patient, which may operate on their own device or be accessed remotely by patient using their own device or a device provided by a medical practitioner. The health information is beneficially provided by the patient answering a questionnaire.

In a third step 120, a personalized prehabilitation program (plan) for the patient is determined based on the received information relating to the medical treatment or procedure and the received health information of the patient. This may be established based on one or more of: heuristic and/or deterministic principles; statistical analysis; and machine learning (for instance (a supervised and/or deep-learning model). Examples of a heuristic approach will be discussed below. A significant portion, the majority or all of the prehabilitation program determination may be performed by the system.

Advantageously, the prehabilitation program comprises a level of support to be provided to the patient. This level of support may indicate one or more of: any base requirements for execution of the program; an indication of communications to be made to the user (for example, in terms of mode of communication, which may include one or more of: calls; text messages; push notifications, and/or content of communication, which may include one or more of: links to services; and curation of available information); and a frequency of program execution and/or follow up.

The determined prehabilitation program is beneficially multimodal and preferably includes one or more of: a physical exercise program; a mental and wellbeing module; nutritional program (guidance and/or advice); mobility exercises; and breathing techniques and exercises. In embodiments, the exercise program comprises one or more of: physical exercises; mobility exercises; and breathing exercises.

In particular, the exercise program may comprise a circuit training prescription. Optionally, the circuit training prescription may form part of the exercise program irrespective of the medical treatment or procedure. At least one parameter of the circuit training prescription may be set based on the received health information. For instance, where the received health information comprises a number of sit to stand movements that the patient can perform independently and without pain and/or discomfort within a 30 second period, the at least one parameter of the circuit training prescription may be set based on the received number of sit to stand movements. In particular, a parameter of the circuit training prescription may be a number of sit to stand exercises, which may be set based on whether the received number of sit to stand movements in the health information exceeds one or more thresholds. The circuit training prescription may comprise one or more of: a hip knees up exercise; a shoulder press exercise; a lateral raise exercise; a knee extension exercise; a knee flexion exercise; a rowing exercise; a hip extension exercise; a hip abduction exercise; and a wall press exercise.

The exercise program may comprise a selection from one or more exercises from a set of exercises for the exercise program, the one or more exercises being selected based on the indication of whether the patient can perform one or more specific activities (as discussed above). The set of exercises may comprise one or more of: shoulder press; bridge; arm lift; and elbow push (which may be selected based on the health information). Optionally, one or more cardiovascular exercises may be set for the exercise program based on the data from the multi-minute walking test. Cardiovascular exercises may include those involving substantial motion of the person (for example, walking, running, jogging) and/or exercises that cause a significant increase in a heart rate of the person from a resting heart rate.

The exercise program may include one of more condition-specific exercises (that is, exercises that are prescribed depending on the condition for which the patient is being treated), for instance one or more of: a pelvic floor exercise; a bracing exercise; a side plank exercise; a plank exercise; a bridge exercise; a shoulder shrug exercise; a shoulder circling exercise; a bent arm forward exercise; a bent arm sideways exercise; a back scratching exercise; a winging it exercise; a wall climbing exercise; an arm lift exercise; and an elbow push exercise. The one of more condition-specific exercises may be selected based on the received information relating to the medical treatment or procedure.

The nutritional program may comprise a recommended protein intake range, for example upper and/or lower limits of daily protein intake. The recommended protein intake range (upper and/or lower limits) may be set based on the received indication of a height and/or weight for the patient, for example based on an ideal body weight (calculated based on height).

Once the prehabilitation program is determined, the determined program is provided or presented to the patient by the system (or processor). In particular, this may be performed by a direct communication (as discussed below). For example, the program may be accessed by the patient using an application on their own device or a device provided by a medical practitioner.

The next stage of the process is termed an intervention period 130 and covers the execution of the determined prehabilitation program by the patient. A length of the intervention period 130 may be determined as part of the prehabilitation program determination and may be unique to each patient, for example based on their baseline assessment. The intervention period 130 comprises two steps: a fourth step 140, in which performance of the determined prehabilitation program is monitored by the system; and an optional fifth step 150, in which the determined prehabilitation program may be adjusted based on results from the monitoring in the fourth step 140.

The fourth step 140, in which performance is monitored, may be performed by the system, in particular with the patient using an application on their device (belonging to them or provided by a medical practitioner), which then communicates with the other parts of the system (for instance, server processor). The monitoring may be manual (input from the user); and/or automatic (based on sensor data or other measurements provided by or through the device, for example using wearable sensor technology).

The system may detect any issues regarding adherence to the program by the patient, for example by comparing the monitored data with the program. The fourth step 140 may further involve, based on the results of the comparison, sending of additional questions (a questionnaire) or prompts to the patient, which may assist in adherence to the program. As part of the monitoring, the application used by the patient may also act as a companion to the patient, for example providing guidance in the form of one or more of documents, video and audio files. The system can add, remove or update documents, so that the feedback given to the patient is up to date.

In the optional fifth step 150, the prehabilitation program is adjusted by the system (or processor). In particular, the adjustment may be based on the monitoring (or comparison) in the fourth step 140. This adjustment may be manual (for example, by a medical practitioner) or automatic, for example based on similar considerations as used for determination of the prehabilitation program in the third step 120.

Subsequent to the intervention period 130, an optional sixth step 160 may provide a follow-on program to the patient. The follow-on program may be determined based on similar considerations as used for determination of the prehabilitation program in the third step 120, for example. The aim of the follow-on program may be to cause the patient to maintain the same level of readiness for the medical treatment or procedure.

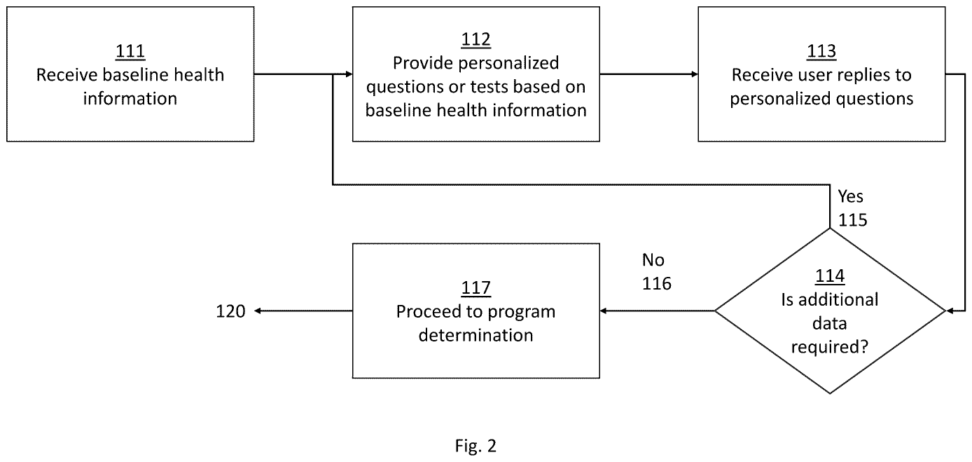

Referring next to FIG. 2, there is shown a schematic flowchart of a computer implemented method according to an embodiment. This shows one implementation of health information gathering in the second step 110 of the method of FIG. 1. As with FIG. 1, the disclosure of FIG. 2 may be implemented at a processor or processor system, for example in a computer server, which will generically be termed a system.

In an initial process, baseline health information is received 111 at the system. This information may be gathered based on a range of questions, which are the same for all patients. The questions may include physical characteristics and/or test results (as discussed above). Based on the answers received in the baseline health information step 111, a personalized set of questions 112 for the patient is provided to the user. Replies to the personalized set of questions are then received in the next step 113. Based on the replies, the system can assess 114 if additional data is required. If the answer is yes 115, further personalized set of questions 112 is provided and this process repeated. If the answer is no 116, the process proceeds in a further step 117 to the third step 120 in the method of FIG. 1, in which the prehabilitation program is determined.

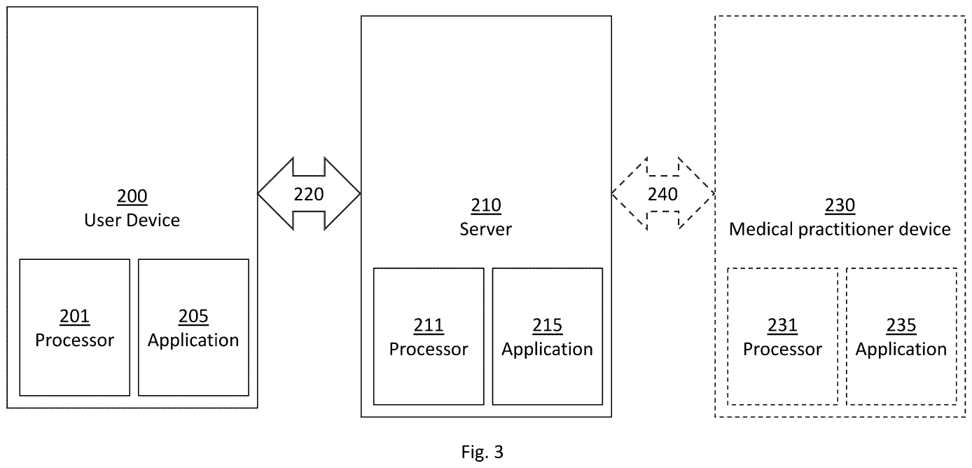

Reference is now made to FIG. 3, in which there is shown a computer system according to an embodiment. The computer system (or variants thereof) may be used for providing a prehabilitation program for a patient prior to undergoing a medical treatment or procedure, in particular for performance of the methods discussed with reference to FIG. 1 and/or FIG. 2.

A user device 200 may be provided to the patient or another user who can obtain information from the patient. As discussed above, the user device may be owned by the user or be a device provided by a medical practitioner to the user for the purposes of the prehabilitation program. The user device 200 comprises a processor 201. An application 205 is stored on the user device 200, for use in performing the method discussed herein. This user-based processor 201 may thus be configured to receive health information of the patient, wherein the health information being indicative of a current state of health for the patient.

A server 210 is operated for the purpose of determining and running one or more prehabilitation programs. The server 210 comprises: a processor 211; and an application 215 stored on the server 210. The application 215 is for use in performing the method discussed herein. The user device 200 and server 210 communicate via an interface 220, which may include a direct interface, an Internet-based interface or a network-specific interface (for example, via a wireless LAN or other network). Thus, the server-based processor 211 may be configured to receive the health information from the user-based processor, to receive information relating to the medical treatment or procedure and to determine the prehabilitation program for the patient based on the received health information and the information relating to the medical treatment or procedure.

Optionally, a medical practitioner device 230 also forms part of the system. The medical practitioner device 230 comprises: a processor 231; and an application 235 stored on the medical practitioner device 230. The application 235 is for use in performing the method discussed herein and may replace the user device 200 and/or server 210 in parts of the method. Advantageously, the medical practitioner device 230 communicates with the server 210 via a second interface 240, which may include a direct interface, an Internet-based interface or a network-specific interface (for example, via a wireless LAN or other network). Typically, the medical practitioner device 230 would not communicate directly with the user device 200, but this may be possible in some circumstances.

Examples of specific prehabilitation programs will now be discussed with reference to physical and/or functional capacity dimensions.

A “Level 1” program may be determined for a patient that requires personal care and needs additional help. Such a patient may have: VO2 Peak<6 ml/kg/min; 30STS<4; and limited mobility outside the house.

A “Level 2” program may be determined for a patient that requires personal care, but is otherwise independent and may be able to perform some household activities (Cleaning, washing, cooking), but needs additional help. Such a patient may have: VO2 Peak 6-8 ml/kg/min; 30STS>4-7; no exercise at all; and able to mobilize inside a house with walking sticks or Zimmer frames. For such a patient, telehealth exercises should only be prescribed after completion of a safety questionnaire and weekly follow-up is then required.

A “Level 3” program may be determined for a patient that requires personal care, but is otherwise independent and may be able to perform household and outdoor activities (walking to shops, gardening, mowing the lawn, using vacuum cleaner). Such a patient may have: VO2 Peak 8-11 ml/kg/min; 30STS>8-11; capable of gentle exercise (for instance, dog walking) most days a week; and able to walk briskly but limited with shortness of breath at short distances (100 m). For such a patient, follow-up should be provided once every two weeks.

A “Level 4” program may be determined for a patient that requires personal care, but is otherwise independent and may be able to perform household and outdoor activities (walking to shops, gardening, mowing the lawn, using vacuum cleaner). Such a patient may have: VO2 Peak 12-15 ml/kg/min; 30STS>12; capable of exercise including (for instance) yoga, Pilates, brisk walking, jogging most days a week. For such a patient, follow-up should be provided once every two weeks.

A “Level 5” program may be determined for a patient that requires personal care, but is otherwise independent and may be able to perform household and outdoor activities (walking to shops, gardening, mowing the lawn, using vacuum cleaner). Such a patient may have: VO2 Peak>15 ml/kg/min; 30STS>12; capable of exercise including (for instance) regular running, weights or resistance training (RT). For such a patient, follow-up should be provided once every month.

A mental assessment may include an assessment of health-related quality of life (HRQOL), which may be termed a well-being scale. This may be assessed by the patient giving themselves a score based on their reflection on their quality of life out of 10. A “Level 3” program may be determined for a score between 10 and 8 (in which case, the application-based resources discussed above may be sufficient), a “Level 2” program may be determined for a score between 8 and 6 (for which an invitation to Tai Chi, Yoga and/or other relaxation sessions may be extended) and a “Level 1” program may be determined for a score below 6 (for which a referral to a counsellor should be provided).

The baseline assessment may also consider nutrition. The health information gathering in the second step 110 of the method of FIG. 1 (and specifically, the personalized set of questions 112 in the method of FIG. 2) may include the following information requests:
1. Has there been any unplanned weight loss in the last 3 months?
2. Has a specialist diet been recommended?
3. Has there been any loss of appetite in the last 3 months?
   If all questions are answered “yes”, a referral to a dietitian should be made. If two questions are answered “yes”, weight and dietary compliance may be monitored via the user application. If one question is answered “yes”, advice should be provided based on guidance with special attention to protein optimization. Where no questions are answered “yes”, the application-based resources discussed above may be sufficient.

Further details of the health information gathering in the second step 110 and the determination process in the third step 120 will now be discussed with reference to an exercise program portion of the prehabilitation program.

The health information gathering in the second step 110 of the method of FIG. 1 (and specifically, the personalized set of questions 112 in the method of FIG. 2) may include the following information requests:
1. Can you do these activities without assistance free of pain/discomfort independently (YES/NO)
   a. sit and stand from a chair?
   b. extend your arms over your head?
   c. lay down flat on the floor and stand up?
2. How many sit-to-stand movements can you perform in a 30 second period (this number is referenced as 30STS below)?

Another test that may be performed as part of the health information gathering in the second step 110 of the method of FIG. 1 is a 6-minute walk test. Typically, the test is time-limited and the person being tested walks as quickly as possible for the stipulated length of time (typically 6 minutes, which is used in the determination below, but the exact length may be not strictly important). Typically, a walking speed is determined, based on a calculated average movement speed from the test. In configuring this test, a number of variables may be identified: a track distance (for example, in meters); a number of laps; a number of stops during the test (n). From these variables, two parameters can be defined: a 6-minute walk distance (6MWD) (m)=Track distance*2*Number of laps; and an average speed (for example, in kilometers/hour)=6MWD/100. The 6MWD may be useful in tailoring a cardiovascular part of the exercise program. Possible example results from the test and the determination of a walking speed from these are discussed below.

| | Track | Treadmill/step counter |
|---|---|---|
| No stops | 3 intervals of 10 min | |
| | 2 min rest between intervals | |
| | 34 min in total | |
| | $Laps / interval = \frac{Number of laps}{6} * 10 * 0.8$ | Walking speed = Average speed * 0.8 |
| | $Laps / minute = \frac{Laps / interval}{10}$ | |
| 1 | 6 intervals of 3 min | |
| | 1 min rest between intervals | |
| | 23 min in total | |
| | $Laps / interval = \frac{Number of laps}{6} * 3 * 0.8$ | Walking speed = Average speed * 0.8 |
| | $Laps / minute = \frac{Laps / interval}{3}$ | |
| 2 | 8 intervals of 2 min | |
| | 0.5 min rest between intervals | |
| | 20 min in total | |
| | $Laps / interval = \frac{Number of laps}{6} * 2 * 0.8$ | Walking speed = Average speed * 0.8 |
| | $Laps / minute = \frac{Laps / interval}{2}$ | |

Based on the answers provided to these questions, an exercise program may be determined. The exercise program comprises a basic circuit (series of exercises), which form part of the exercise program for all patients (although portions of the exercise program may be tailored according to the answers provided to the above questions) and a treatment-specific part, which depends on the medical treatment or procedure for which the patient is requesting prehabilitation). A sample exercise program structure is provided below.

| | | | |
|---|---|---|---|
| Basic circuit | | 1a = true | 1a = false |
| (everyone) | 1 | Sit-to-stand | Empty |
| Twice a week | | If 30STS < 8 then | |
| on alternate | | If 30STS-30STS* | |
| days | | 0.5 ≥ 1 then | |
| 2 rounds of | | 30STS-30STS*0.5 | |
| exercises 1 to | | Else | |
| 10 | | Empty | |
| 10 repetitions | | Else | |
| per exercise | | 30STS-30STS*0.2 | |
| | 2 | Upright row | |
| | 3 | Hip knees up | |
| | | 1b = true | 1b = false |
| | 4 | Shoulder press | Lateral |
| | | | raises |
| | 5 | Knee extension | |
| | 6 | Knee flexion | |
| | 7 | Row | |
| | 8 | Hip extension | |
| | 9 | Hip abduction | |
| | 10 | Wall press | |
| Prostate cancer | | Pelvic floor exercises | |
| Twice a day | | | |
| Colorectal cancer | 1 | Bracing | |
| Once a day | 2 | Side plank | |
| 3 rounds of | 3 | Plank | |
| exercises 1 to 4 | | 1c = true | 1c = false |
| 10 seconds/ | 4 | Bridge | |
| exercise | | | |
| Breast cancer | 1 | Shoulder shrugs | |
| Three times a day | 2 | Shoulder circling | |
| Exercises 1 to 11 | 3 | Bent arm forward | |
| 5 repetitions | 4 | Bent arm sideways | |
| per exercise | 5 | Back scratching | |
| | 6 | Winging it | |
| | 7 | Wall climbing | |
| | | 1c = true | 1c = false |
| | 8 | Arm lifts Empty | |
| | 9 | Elbow push | Empty |
| | 10 | Shoulder shrugs | |
| | 11 | Shoulder circling | |
| Lung cancer | | As per instructions in | |
| | | Inspiratory Muscle | |
| | | Training (below) | |

The exercise program may also include inspiratory muscle training. This training has two variables: maximum inspiratory pressure (MIP) (cmH₂O); and forced vital capacity (FVC) (ml).

If MIP and pressure regulated inspiratory trainer are available

| | |
|---|---|
| Prehab duration 1-2 weeks | Prehab duration |
| (Ma 2021) | >2 weeks (Morano |
| | 2013) |
| Initial training pressure | Initial training |
| 30% MIP | pressure 20% MIP |
| 15 min | 10 min |
| Twice a day | Twice a day |
| Daily 5% increase until | Daily 5% increase |
| maximum tolerable | until maximum tolerable |

If FVC and a volume-oriented incentive
spirometer are available (Sebio Garcia 2017)

6 cycles of 5 inspirations
1 min rest between cycles
30 inspirations in total
Twice a day

The prehabilitation program may further include a nutritional component. As part of this, a recommended protein intake range may be set, in particular based on a height of the patient. A recommended lower limit protein intake may be set as 1.2 g×ideal body weight and a recommended upper limit protein intake may be set as 1.5 g×ideal body weight. This may result in the following formulae:

$$recommended lower limit protein intake ( g ) = 1.2 * 19.5 * {height ( m )}^{2} ; and$$

$$recommended upper limit protein intake ( g ) = 1.5 * 24.9 * {height ( m )}^{2} .$$

Implementations according to the disclosure may be in terms of software, for example as a computer program product. Such a computer program product may be embodied on a (non-transitory) computer readable medium.

Although a number of embodiments have been described, the skilled person will appreciate that modifications and variations are possible. For example, the exact format of the prehabilitation program may be varied and/or the system architecture may change to reflect technological implementation and/or to be more centralized or distributed according to circumstances.

As used herein, including in the claims, unless the context indicates otherwise, singular forms of the terms herein are to be construed as including the plural form and vice versa. For instance, unless the context indicates otherwise, a singular reference herein including in the claims, such as “a” or “an” means “one or more”. Throughout the description and claims of this disclosure, the words “comprise”, “including”, “having” and “contain” and variations of the words, for example “comprising” and “comprises” or similar, mean “including but not limited to”, and are not intended to (and do not) exclude other components.

The use of any and all examples, or exemplary language (“for instance”, “such as”, “for example” and like language) provided herein, is intended merely to better illustrate the invention and does not indicate a limitation on the scope of the invention unless otherwise claimed. No language in the specification should be construed as indicating any non-claimed element as essential to the practice of the invention.

Any steps described in this specification may be performed in any order or simultaneously unless stated or the context requires otherwise.

All of the aspects and/or features disclosed in this specification may be combined in any combination, except combinations where at least some of such features and/or steps are mutually exclusive. As described herein, there may be particular combinations of aspects that are of further benefit. In particular, the preferred features of the invention are applicable to all aspects of the invention and may be used in any combination. Likewise, features described in non-essential combinations may be used separately (not in combination).

1. A computer implemented method for providing a prehabilitation program for a patient prior to undergoing a medical treatment or procedure, the method comprising:
receiving at a processor, health information of the patient and information relating to the medical treatment or procedure, the health information being indicative of a current state of health for the patient; and
determining, at the processor, the prehabilitation program for the patient based on the received health information and the information relating to the medical treatment or procedure.

2. The computer implemented method of claim 1, wherein the health information comprises information indicative of a current state of physical and mental health for the patient and wherein the prehabilitation program comprises a level of support to be provided to the patient.

3. The computer implemented method of claim 1, wherein the prehabilitation program comprises an exercise program and/or a nutrition program.

4. The computer implemented method of claim 3, wherein the step of determining comprises:
determining upper and lower limits of daily protein intake.

5. The method of claim 3, wherein the received health information comprises an indication of whether the patient can perform one or more specific activities independently and without pain and/or discomfort, and wherein the step of determining comprises selecting one or more exercises from a set of exercises for the exercise program, the one or more exercises being selected based on the indication of whether the patient can perform the one or more specific activities.

6. The method of claim 5, wherein the specific activities comprise one or more of: sitting and standing from a chair, extending arms over head, laying down flat on the floor and standing up and/or wherein the set of exercises comprise one or more of: shoulder press; bridge; arm lift; and elbow push.

7. The computer implemented method of claim 3, wherein the exercise program comprises a circuit training prescription, at least one parameter of the circuit training prescription being set based on the received health information.

8. The computer implemented method of claim 7, wherein the received health information comprises a number of sit to stand movements that the patient can perform independently and without pain and/or discomfort within a 30 second period, and wherein the at least one parameter of the circuit training prescription is set based on the received number of sit to stand movements.

9. The computer implemented method of claim 8, wherein the at least one parameter of the circuit training prescription comprises a number of sit to stand exercises and wherein the number of sit to stand exercises is set based on whether the received number of sit to stand movements exceeds one or more thresholds.

10. The computer implemented method of claim 7, wherein the exercise program comprises a circuit training prescription irrespective of the medical treatment or procedure.

11. The computer implemented method of claim 7, wherein the circuit training prescription comprises one or more of: a hip knees up exercise; a shoulder press exercise; a lateral raise exercise; a knee extension exercise; a knee flexion exercise; a rowing exercise; a hip extension exercise; a hip abduction exercise; and a wall press exercise.

12. The computer implemented method of claim 3, wherein the exercise program comprises one of more condition-specific exercises, the one of more condition-specific exercises comprising one or more of: a pelvic floor exercise; a bracing exercise; a side plank exercise; a plank exercise; a bridge exercise; a shoulder shrug exercise; a shoulder circling exercise; a bent arm forward exercise; a bent arm sideways exercise; a back scratching exercise; a winging it exercise; a wall climbing exercise; an arm lift exercise; and an elbow push exercise, the one of more condition-specific exercises being selected based on the received information relating to the medical treatment or procedure.

13. The computer implemented method of claim 3, wherein the received health information comprises data from a multi-minute walking test undertaken by the patient, and wherein the step of determining comprises setting one or more cardiovascular exercises for the exercise program based on the data from the multi-minute walking test.

14. The computer implemented method of claim 3, wherein the exercise program comprises one or more of: physical exercises; mobility exercises; and breathing exercises.

15. The computer implemented method of claim 3, wherein the health information comprises an indication of a height and/or weight for the patient, and wherein the nutrition program comprises a recommended protein intake range that is set based on the received indication of a height and/or weight for the patient.

16. The computer implemented method of claim 1, further comprising:
presenting the prehabilitation program to the patient by the processor.

17. The computer implemented method of claim 1, further comprising:
monitoring performance of the prehabilitation program by the patient at the processor.

18. The computer implemented method of claim 17, wherein the step of monitoring is based on user-input information and/or sensor-based information and/or wherein the method further comprises adjusting the prehabilitation program by the processor based on the monitoring.

19. A computer system for providing a prehabilitation program for a patient prior to undergoing a medical treatment or procedure, the computer system comprising at least one processor, configured to:
receive health information of the patient and information relating to the medical treatment or procedure, the health information being indicative of a current state of health for the patient; and
determine the prehabilitation program for the patient based on the received health information and the information relating to the medical treatment or procedure.

20. A computer system for providing a prehabilitation program for a patient prior to undergoing a medical treatment or procedure, the computer system comprising:
a user-based processor, configured to receive health information of the patient, the health information being indicative of a current state of health for the patient; and
a server-based processor, configured to receive the health information from the user-based processor, to receive information relating to the medical treatment or procedure and to determine the prehabilitation program for the patient based on the received health information and the information relating to the medical treatment or procedure.
